(19) United States
(12) Patent Application Publication
(10) Pub. No.: US 2026/0356906 A1
(43) Pub. Date: Nov. 12, 2026

(54) AAV-MEDIATED INTRAMUSCULAR DELIVERY OF INSULIN

(71) Applicant: AAV Gene Therapeutics, Inc., Beverly, MA (US)

(72) Inventor: Senthil Ramu, Boston, MA (US)

(21) Appl. No.: 19/100,458

(22) PCT Filed: Aug. 9, 2023
(86) PCT No.: PCT/US2023/029845

§ 371 Date: Jan. 31, 2025

Related U.S. Application Data
(60) Provisional application No. 63/396,861, filed on Aug. 10, 2022.

(51) Int. Cl. C07K 14/62; A61K 38/00; A61K 48/00; A61P 3/10; C12N 15/86

(52) U.S. Cl. CPC C07K 14/62; A61K 48/0058; A61P 3/10; C12N 15/86; A61K 38/00; C07K 2319/02; C07K 2319/50; C12N 2750/14143

(57) ABSTRACT

The invention described herein provides an engineered insulin polypeptide comprising two cleavable peptides interposed between the insulin B-chain and the C-peptide, and between the C-peptide and the insulin A-chain to secrete active insulin from muscle cells. Also provides are polynucleotide encoding the engineered insulin for intramuscular delivery through Adeno Associated Virus (AAV) vectors, and the use thereof to treat diseases such as diabetes, hyperglycemia and other hyperglycemia-related diseases.

### REFERENCE TO RELATED APPLICATION

This application claims priority to and the benefit of the filing date of U.S. Provisional Patent Application No. 63/396,861, filed on Aug. 10, 2022, the entire contents of which (including any drawings and sequences) are incorporated herein by reference.

### BACKGROUND OF THE INVENTION

Eukaryotic secreted proteins are generally processed and secreted via either the constitutive pathway or the regulated pathway.

Insulin is one of many secreted proteins in mammals. It is synthesized as an inactive preproinsulin having a signal peptide. The preproinsulin is translocated directly into the rough endoplasmic reticulum (RER), where its signal peptide is removed by signal peptidase to form proinsulin. As the proinsulin naturally folds, a C-terminal sequence called “A-chain” and an N-terminal sequence called “B-chain” become covalently linked together with three disulfide bonds (with one of the three being an intra-chain disulfide bond linking two Cys residues of the A-chain). This folded proinsulin then transits through the Golgi apparatus, and is packaged into specialized secretory vesicles. Within such granules, proinsulin is cleaved by specialized peptidases called proprotein convertase 1/3 and proprotein convertase 2, removing the middle part of the protein, called the “C-peptide.” Finally, carboxypeptidase E removes two pairs of amino acids from the protein's ends, resulting in active insulin—the insulin A- and B-chains, connected with two disulfide bonds.

This process only occurs in the secretory vesicles of pancreatic β-cells in mammals, which secretory vesicles contain all the requisite processing enzymes described above. In cells with only the constitutive pathway, there are no such specialized proinsulin processing enzymes. Thus any synthesized preproinsulin there (if any) will not be processed into mature insulin for release outside the cells.

### SUMMARY OF THE INVENTION

One aspect of the invention provides an engineered insulin polypeptide (E-insulin) engineered from a wild-type insulin sequence in a mammal, comprising: (a) a first cleavable heterologous peptide, between the insulin B-chain and the insulin C-peptide of the wild-type insulin; and, (b) a second cleavable heterologous peptide, between the insulin C-peptide and the insulin A-chain of the wild-type insulin; wherein the first and the second cleavable heterologous peptides are both native sequences of the mammal.

In certain embodiments, the mammal is a human.

In certain embodiments, the first and the second cleavable heterologous peptides are both cleavable by a serine endoprotease expressed in muscle tissues.

In certain embodiments, the serine endoprotease is a subtilisin-like endoprotease, such as furin, PACE4, PC4, PC5/6, or LPC/PC8.

In certain embodiments, the first cleavable heterologous peptide and the second cleavable heterologous peptide independently comprise a sequence motif Arg-Xaa-Xaa-Arg (wherein Xaa is any amino acid) or Lys/Arg-Arg.

In certain embodiments, the serine endoprotease is present or expressed in trans-Golgi network (TGN), such as TGN of muscle tissue/cell.

In certain embodiments, the serine endoprotease is Furin.

In certain embodiments, the first cleavable heterologous peptide and the second cleavable heterologous peptide both comprise, consist essentially of, or consist of an amino acid sequence independently selected from RGVFRR (SEQ ID NO: 3); RAKR; RKKR; RKYR; RRKR RRRR; RQRR; LSRR; RKRR; RHKR; RERR; RPRR; RARR; RSAR; LRKR; RTRR RTAR; RNTR; RLRR; RWRR; RGKR; RVKR; RHPR; RRGR; RVRR; RDRR; RTGR REAR; RFKR; RYKR; RHAR; RLLR; RVGR; RRAR; HPKR; RFPR; RRTR; RNQR; RSRR RRRK; RSIR; RTPR; RPAR; RNHR; RKTR; RKNR; RLKR; RNRR; RKSR; RMKR; RNKR RQQR; RIRR; REIR; REKR; RMRR; RPDR; RIVR; RPKR; RLKK; RSKR; RVAR; RGPR RVCR; RAPR; RQIR; RYRR; RKIR; RMIR; RVRK; RRVR; RCQR; RCKR; VRKR; RPVR RHTR; RCRR; RGRR; RISR; RAAR; RLGR; RYPR; RAGR; RIIR; KSAR; VSRR; RQCR RVKK; RIGR; RTTR; RHRR; VVAR; RSHR; RYSR; RCIR; RCYR; RFRK; RYFR; RTMR RHGR; RSPR; RETR; RKFR; RHLR; RKGR; RRKK; RWKR; RVLR; RIKR; VKKR; RFRR RALR; MKKR; RVTR; NCSR; KRKR; RSSR; RMAR; KRRR; RRPR; TIKR; HLKR; RFER RASR; RRSR; VRRR; KAKR; RDKR; KVKR; RFLR; RLPR; RPPR; RHSR; RSGR; RICR HKKR; RSLR; RLVR; RVPR; RFSR; RTIR; RLTR; RKAR; RAQR; RQMR; RFVR RSNR; RQPR; CVRR; RSYR; IKKR; RHVR; RLAR; RPLR; IGKR; VTKR; RRHR; RRNR VHKR; RCPR; RWPR; SKER; RIKK; RIFR; RVWR; LEGR; LKKR; VFRR; IRKR.

In certain embodiments, the insulin B-chain comprises, consists essentially of, or consists of the amino acid sequence of SEQ ID NO: 2.

In certain embodiments, the insulin C-peptide comprises, consists essentially of, or consists of the amino acid sequence of SEQ ID NO: 4.

In certain embodiments, the insulin A-chain comprises, consists essentially of, or consists of the amino acid sequence of SEQ ID NO: 5.

In certain embodiments, the E-insulin of the invention further comprises a signal peptide.

In certain embodiments, the signal peptide comprises, consists essentially of, or consists of the amino acid sequence of SEQ ID NO: 1.

In certain embodiments, the insulin A-chain and the insulin B-chain are covalently linked by a disulfide bond.

In certain embodiments, the insulin A-chain and the insulin B-chain are covalently linked by two disulfide bonds.

In certain embodiments, the E-insulin of the invention further comprises an H10D substitution on the insulin B-chain.

Another aspect of the invention provides a mature E-insulin resulting from cleavage of the E-insulin of the invention described herein, at the first cleavable heterologous peptide and said second cleavable heterologous peptide, wherein said insulin A-chain and said insulin B-chain are covalently linked by two disulfide bonds.

In certain embodiments, the mature E-insulin comprises a first remnant sequence of the first cleavable heterologous peptide, optionally, the first remnant sequence (and further optionally the last one or two basic residue(s) at the C-terminus of SEQ ID NO: 2) is removed by a carboxypeptidase (such as carboxypeptidase E) in said mature E-insulin.

Another aspect of the invention provides a cleaved insulin C-peptide resulting from cleavage of the E-insulin of the invention at the first cleavable heterologous peptide and the second cleavable heterologous peptide, wherein the cleaved insulin C-peptide comprises a remnant sequence of the second cleavable heterologous peptide; optionally, the second remnant sequence (and further optionally the last one or two basic residue(s) at the C-terminus of SEQ ID NO: 4) is removed by a carboxypeptidase (such as carboxypeptidase E) in the cleaved insulin C-peptide.

Another aspect of the invention provides a polynucleotide encoding the E-insulin of the invention described herein.

Another aspect of the invention provides a vector comprising the polynucleotide of the invention described herein.

In certain embodiments, the vector comprises a promoter operably linked to a transcription cassette comprising the polynucleotide of the invention.

In certain embodiments, the promoter comprises: (1) a constitutively active eukaryotic promoter, (2) a muscle-specific promoter, and/or (3) a synthetic promoter.

In certain embodiments, the vector comprises two promoters.

In certain embodiments, the vector comprises: (1) a first transcription cassette comprising the polynucleotide of the invention operably linked to a first promoter (e.g., a muscle-specific promoter such as MCK, or a constitutive promoter such as CBA or CAG); and, (2) a second transcription cassette comprising the polynucleotide of the invention operably linked to a second promoter, such as a synthetic promoter; wherein the polynucleotides of the invention in the first and the second transcription cassettes are independently selected from the E-insulin of the invention described herein.

In certain embodiments, the transcription cassette(s) further encodes a 5′-UTR region, a 3′-UTR region, and/or a polyA signal operably linked to the polynucleotide of the invention.

In certain embodiments, the vector is an AAV (adeno-associated viral) vector further comprising a 5′ ITR and/or a 3′ ITR.

Another aspect of the invention provides an AAV viral particle comprising the AAV vector of the invention, encapsidated within a capsid.

In certain embodiments, the capsid has a serotype with muscle tropism, such as tropism for skeletal muscle, smooth muscle, or cardiac muscle.

In certain embodiments, the capsid has the serotype of AAV1, AAV2, AAV6, AAV7, AAV8, AAV9, AAV-DJ/8, AAV-Rh10, AAV-retro, AAV-PHP.B, AAV-PHP.eB, or AAV-PHP.S, or a genetically engineered AAV Capsid.

Another aspect of the invention provides a method of delivering an engineered insulin polypeptide to a muscle cell, the method comprising contacting the muscle cell with the AAV viral particle of the invention.

In certain embodiments, the muscle cell is in an animal, such as a human or a non-human mammal.

In certain embodiments, the animal has Type 1 or Type 2 diabetes, or hyperglycemia.

Another aspect of the invention provides a method of treating a subject in need of treatment for a disease treatable by insulin, the method comprising administering to the subject a therapeutically effective amount of the AAV viral particle of the invention.

In certain embodiments, the AAV viral particle is delivered intramuscularly.

In certain embodiments, the subject has Type 1 diabetes.

In certain embodiments, the subject has Type 2 diabetes.

In certain embodiments, the subject has hyperglycemia.

It should be understood that any one embodiment of any one of the aspects of the invention above, including specific embodiments described only in one section of the application, can be combined with one or more additional embodiments.

### BRIEF DESCRIPTION OF THE DRAWINGS

FIG. 1 is a schematic (not to scale) illustration of the synthesis and processing of one embodiment of the engineered insulin (E-insulin) of the invention.

FIG. 2 is a schematic (not to scale) illustration of a more detailed structure of the one embodiment of the E-insulin of the invention depicted in FIG. 1.

FIG. 3 is a schematic (not to scale) illustration of a wild-type insulin structure for comparison with that of the E-insulin of FIG. 2.

FIG. 4 is an electrophoresis image showing the plasmid constructs encoding with the full-length wild-type (FL) human insulin, or one E-insulin of the invention. The E-insulin insert after restriction enzyme digestion is slightly larger than that of the FL insulin.

FIG. 5 shows that the processed mature insulin (E-insulin) were produced and secreted from mouse C2C12 muscle cells.

FIG. 6 shows that the C-peptide was secreted and released from the E-insulin or FL insulin from mouse C2C12 muscle cells.

FIG. 7 shows potency of secreted E-insulin in muscle cells by eliciting differentiation of C2C12 myoblast muscle cells into myotubes. Immunohistochemical localization of myosin heavy chain is used as molecular marker for myotube differentiation. The DAPI standing shows the locations of the muscle cell nuclei.

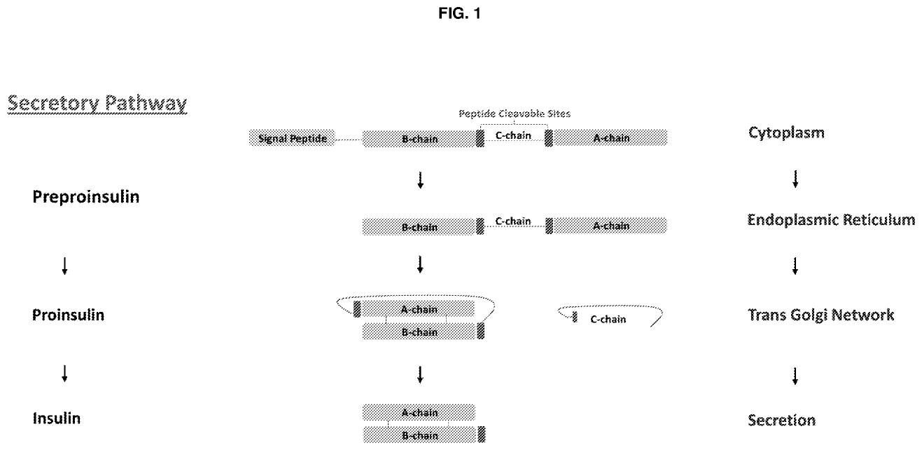

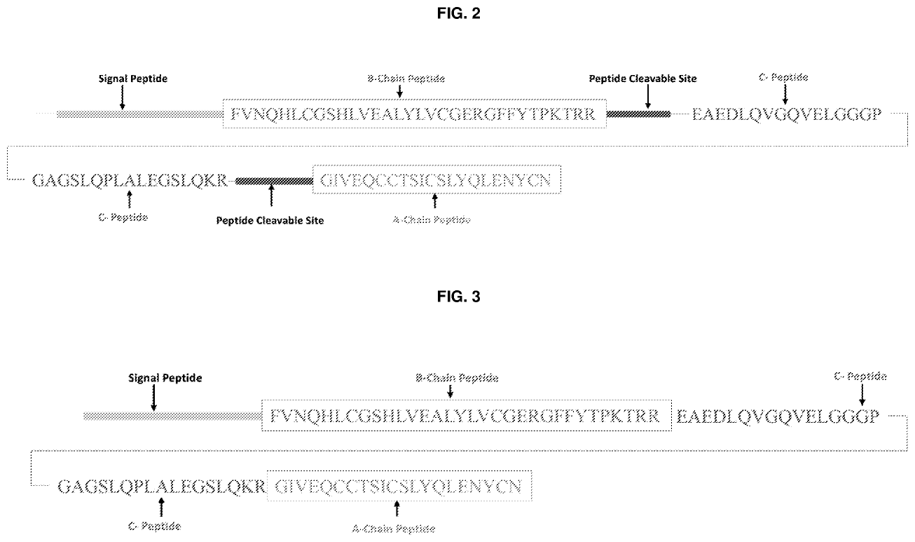

### DETAILED DESCRIPTION OF THE INVENTION

### 1. Overview

The invention described herein provides an engineered insulin (E-insulin) construct and the E-insulin encoded thereby, wherein the E-insulin can be processed in vivo, in cells (such as muscle cells) that do not otherwise process wild-type preproinsulin to mature/active insulin. The invention is partly based on the introduction of two cleavable heterologous peptides that flanks the C-peptide in the E-insulin precursor, such that such cleavable heterologous peptides can be cleaved in the cells (e.g., muscle cells) to release mature insulin.

One salient feature of the E-insulin of the invention is that such cleavable heterologous peptides are both native sequences of the mammal. While not wishing to be bound by any particular theory, it is believed that the E-insulin with such cleavable heterologous peptides has reduced risk (if not without any risk) of causing host immune response, such as T- and B-cell mediated cellular and humoral immune responses against the E-insulin as both are native sequence of the mammal.

Thus, one aspect of the invention provides an engineered insulin polypeptide (E-insulin) engineered from a wild-type insulin sequence in a mammal, comprising: (a) a first cleavable heterologous peptide, between the insulin B-chain and the insulin C-peptide of the wild-type insulin; and, (b) a second cleavable heterologous peptide, between the insulin C-peptide and the insulin A-chain of the wild-type insulin; wherein the first and the second cleavable heterologous peptides are both native sequences of the mammal.

As used herein, “native sequences of the mammal” includes naturally existing sequences in the mammal. Such naturally existing sequences are unlikely to trigger undesirable host animal immune response, since such sequences are deemed self-antigens with respect to the host animal.

In certain embodiments, the mammal is a human. In certain embodiments, the mammal is a non-human animal, such as a rodent (such as a mouse, a rat, a rabbit, a hamster, a guinea pig, etc), pets (e.g., all breeds of dogs and cats and pet mammals), a livestock mammal (such as a horse, a sheep, a goat, a pig, a cow/bull), or a non-human primate, such as a monkey (e.g., a cynomolgus monkey, a rhesus monkey etc).

According to the invention, the E-insulin is engineered from the wild-type (wt) insulin of the mammal. For example, an E-insulin of the invention for human is based on/engineered from the wild-type human insulin sequence, and the cleavable heterologous peptides are also from human.

The cleavable heterologous peptides are “heterologous,” partly because these sequences/peptides do not originate from insulin or its precursor thereof.

Any cleavable heterologous peptides may be used in the E-insulin of the invention, provided that they are cleavable by an enzyme expressed inside the target cell in which such E-insulin is to be expressed.

In certain embodiments, the first and the second cleavable heterologous peptides are both cleavable by a serine endoprotease expressed in muscle tissues.

For example, in certain embodiments, the serine endoprotease is a subtilisin-like endoprotease, such as furin, PACE4, PC4, PC5/6, or LPC/PC8.

In certain embodiments, the first cleavable heterologous peptide and the second cleavable heterologous peptide independently comprise a sequence motif Arg-Xaa-Xaa-Arg (wherein Xaa is any amino acid) or Lys/Arg-Arg.

In certain embodiments, the serine endoprotease is present or expressed in trans-Golgi network (TGN), such as TGN of muscle tissue/cell.

In certain embodiments, the serine endoprotease is Furin.

Furin is also known as PACE (Paired basic Amino acid Cleaving Enzyme). It is a member of family S8, and is a member of the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. The furin substrates and the locations of furin cleavage sites in protein sequences can be predicted by two bioinformatics methods: ProP (Duckert et al., Protein Engineering, Design & Selection 17 (1): 107-112, 2004) and PiTou (Tian et al., Scientific Reports. 2: 261, 2012), both incorporated herein by reference.

In certain embodiments, the first cleavable heterologous peptide and the second cleavable heterologous peptide both comprise, consist essentially of, or consist of an amino acid sequence independently selected from RGVFRR (SEQ ID NO: 3), RAKR; RKKR; RKYR; RRKR RRRR; RQRR; LSRR; RKRR; RHKR; RERR; RPRR; RARR; RSAR; LRKR; RTRR RTAR; RNTR; RLRR; RWRR; RGKR; RVKR; RHPR; RRGR; RVRR; RDRR; RTGR REAR; RFKR; RYKR; RHAR; RLLR; RVGR; RRAR; HPKR; RFPR; RRTR; RNQR; RSRR RRRK; RSIR; RTPR; RPAR; RNHR; RKTR; RKNR; RLKR; RNRR; RKSR; RMKR; RNKR RQQR; RIRR; REIR; REKR; RMRR; RPDR; RIVR; RPKR; RLKK; RSKR; RVAR; RGPR RVCR; RAPR; RQIR; RYRR; RKIR; RMIR; RVRK; RRVR; RCQR; RCKR; VRKR; RPVR RHTR; RCRR; RGRR; RISR; RAAR; RLGR; RYPR; RAGR; RIIR; KSAR; VSRR; RQCR RVKK; RIGR; RTTR; RHRR; VVAR; RSHR; RYSR; RCIR; RCYR; RFRK; RYFR; RTMR RHGR; RSPR; RETR; RKFR; RHLR; RKGR; RRKK; RWKR; RVLR; RIKR; VKKR; RFRR RALR; MKKR; RVTR; NCSR; KRKR; RSSR; RMAR; KRRR; RRPR; TIKR; HLKR; RFER RASR; RRSR; VRRR; KAKR; RDKR; KVKR; RFLR; RLPR; RPPR; RHSR; RSGR; RICR HKKR; RSLR; RLVR; RVPR; RFSR; RTIR; RLTR; RKAR; RAQR; RQMR; RFVR RSNR; RQPR; CVRR; RSYR; IKKR; RHVR; RLAR; RPLR; IGKR; VTKR; RRHR; RRNR VHKR; RCPR; RWPR; SKER; RIKK; RIFR; RVWR; LEGR; LKKR; VFRR; and IRKR.

In certain embodiments, the first cleavable heterologous peptide and/or the second cleavable heterologous peptide are not RQKR, RTKR, LQKR, KTRR, KTKR, and KTRR.

In certain embodiments, the insulin B-chain comprises, consists essentially of, or consists of the amino acid sequence of SEQ ID NO: 2.

In certain embodiments, the insulin C-peptide comprises, consists essentially of, or consists of the amino acid sequence of SEQ ID NO: 4.

In certain embodiments, the insulin A-chain comprises, consists essentially of, or consists of the amino acid sequence of SEQ ID NO: 5.

In certain embodiments, the E-insulin of the invention further comprises a signal peptide.

In certain embodiments, the signal peptide comprises, consists essentially of, or consists of the amino acid sequence of SEQ ID NO: 1.

In certain embodiments, the E-insulin comprises, consists essentially of, or consists of the amino acid sequence of SEQ ID NO: 7. In certain embodiments, the E-insulin is a variant of SEQ ID NO: 7 having at most 1, 2, 3, 4, 5, 6, 7, 8, 9, or 10 changes (deletions, insertions, substitutions) that do not substantially alter (e.g., decrease no more than 5%, 10%, 15%, 20% of the E-insulin or wt insulin activity).

In certain embodiments, the E-insulin comprises, consists essentially of, or consists of the amino acid sequence encoded by SEQ ID NO: 6.

In certain embodiments, the insulin A-chain and the insulin B-chain are covalently linked by a disulfide bond.

In certain embodiments, the insulin A-chain and the insulin B-chain are covalently linked by two disulfide bonds.

In certain embodiments, the E-insulin of the invention further comprises an H10D substitution on the insulin B-chain.

Another aspect of the invention provides a mature E-insulin resulting from cleavage of the E-insulin of the invention described herein, at the first cleavable heterologous peptide and said second cleavable heterologous peptide, wherein said insulin A-chain and said insulin B-chain are covalently linked by two disulfide bonds.

In certain embodiments, the mature E-insulin comprises a first remnant sequence of the first cleavable heterologous peptide, optionally, the first remnant sequence is removed by a carboxypeptidase (such as carboxypeptidase E) in said mature E-insulin.

In certain embodiments, the mature E-insulin does not have any remnant sequence of the first cleavable heterologous peptide after removal of such remnant sequence by a carboxypeptidase (such as carboxypeptidase E).

Another aspect of the invention provides a cleaved insulin C-peptide resulting from cleavage of the E-insulin of the invention at the first cleavable heterologous peptide and the second cleavable heterologous peptide, wherein the cleaved insulin C-peptide comprises a remnant sequence of the second cleavable heterologous peptide; optionally, the second remnant sequence is removed by a carboxypeptidase (such as carboxypeptidase E) in the cleaved insulin C-peptide.

Another aspect of the invention provides a polynucleotide encoding the E-insulin of the invention described herein.

Another aspect of the invention provides a vector comprising the polynucleotide of the invention described herein.

In certain embodiments, the vector comprises a transcriptional regulatory element, such as a promoter and/or enhancer, operably linked to a transcription cassette comprising the polynucleotide of the invention.

In certain embodiments, the enhancer and/or the promoter is a muscle-specific control element.

The term “muscle specific control element” refers to a nucleotide sequence that regulates expression of a coding sequence that is specific for expression in muscle tissue. These control elements include enhancers and/or promoters. Exemplary muscle specific control elements include a MCKH7 promoter, an MCK promoter, and a MCK enhancer.

Other muscle-specific control element include human skeletal actin gene element, cardiac actin gene element, myocyte-specific enhancer binding factor MEF, muscle creatine kinase (MCK), tMCK (truncated MCK), myosin heavy chain (MHC), MHCK7 (a hybrid version of MHC and MCK), C5-12 (synthetic promoter), murine creatine kinase enhancer element, skeletal fast-twitch troponin C gene element, slow-twitch cardiac troponin C gene element, slow-twitch troponin I gene element, hypoxia-inducible nuclear factors, steroid-inducible element or glucocorticoid response element (GRE).

In certain embodiments, the promoter comprises: (1) a constitutively active eukaryotic promoter, (2) a muscle-specific promoter, and/or (3) a synthetic promoter.

In certain embodiments, the vector comprises two promoters.

In certain embodiments, the vector comprises: (1) a first transcription cassette comprising the polynucleotide of the invention operably linked to a first promoter (e.g., a muscle-specific promoter such as MCK, or a constitutive promoter such as CBA or CAG); and, (2) a second transcription cassette comprising the polynucleotide of the invention operably linked to a second promoter, such as a synthetic promoter; wherein the polynucleotides of the invention in the first and the second transcription cassettes are independently selected from the E-insulin of the invention described herein.

In certain embodiments, the muscle-specific promoter is MHCK7, CK8e, tMCK, (human) skeletal α-actin (HSA) promoter (including full length or shortened HAS promoter, and chimeric HSA/CMV promoter consisting of a fragment of the HSA promoter and the CMV promoter), muscle creatine kinase (MCK/CKM) promoter or derivative thereof (including CK6, MHCK7, dMCK, tMCK, CK8 and CK8e), desmin gene promoter, human α-myosin heavy chain gene (aMHC) promoter, myosin light-chain promoter (MLC2v), cardiac troponin T promoter (cTnT), a chimeric promoter comprising the CMV-IE enhancer ligated to the 1.5-kbp fragment of the rat promoter MLC, and the ΔUSEx3 promoter developed from the human troponin I (TNNI1) gene. See Skopenkova et al., Muscle-Specific Promoters for Gene Therapy, Acta Naturae. 13(1): 47-58, 2021 (incorporated herein by reference).

In certain embodiments, the constitutive promoter is: CAG, CB, CB6, respiratory syncytial virus (RSV) promoter, cytomegalovirus (CMV) promoter, or elongation factor 1a (EF1a) promoter.

In certain embodiments, the promoter is a synthetic promoter. Synthetic promoter is a groundbreaking approach in promoter design. This strategy enables one to engineer promoters with defined properties, such as size and the expression profile of the transgene. The development of synthetic promoters relies on computational algorithms, which are used to identify regulatory sequences and TFBSs within the genome, as well as to predict the promoter regions. For example, the binding sites for myogenic TFs are usually shorter than 10 bp, which allows one to create a library of constructs with different combinations of muscle-specific TFBSs.

In certain embodiments, the synthetic promoter is the SPc5-12 promoter (Li et al., Nat. Biotechnol. 17(3):241-245, 1999). The SPc5-12 promoter consists of a combination of four muscle-specific TFBSs (TEF1, SRE, MEF1, and MEF2) and the core promoter (a fragment of the promoter of the chicken skeletal muscle α-actin gene). Its activity in muscle fibers is reportedly six-fold higher than that of the CMV promoter. Further, in vivo experiments confirmed that the SPc5-12 promoter is inactive in undifferentiated myoblasts and in various nonmuscle cell lines.

In certain embodiments, the synthetic promoter is the SP-301 promoter (Liu et al., Plasmid. 106:102441, 2019). The SP-301 promoter is a combination of muscle-specific TFBSs, viral elements, and conserved cis-regulatory elements ligated in forward and reverse orientation. The SP-301 promoter is 6.6 times more active than the CMV promoter 2 days after intramuscular delivery of the construct in mice and remained active for at least a month. The tissue specificity of the SP-301 promoter was confirmed in transgenic mice.

In certain embodiments, the synthetic promoter is the MH promoter. The MH promoter consists of the human desmin gene enhancer linked to the enhancer, the core promoter, and the first intron of the mouse Ckm gene. The MH promoter ensured the highest expression level in the muscle cell culture, being superior to the desmin and CMV promoters. AAV2/9 carrying a reporter gene delivered intravenously in mice under the control of the MH promoter has higher reporter activity in the cardiac and skeletal muscles than that of the desmin and CMV promoters. Further, the MH promoter does not induce transgene expression in the liver.

In certain embodiments, the synthetic promoter is the Sk-CRM4/Des promoter, which is the regulatory module Sk-CRM4 ligated to the desmin promoter and the MVM intron. Six weeks after systemic delivery using AAV9, the Sk-CRM4 chimeric promoter enhanced the activity of the desmin promoter by 200-400 times in different skeletal muscles, the diaphragm, and the heart, while remaining inactive in non-target tissues. Moreover, the SkCRM4/Des promoter attained a 25-173 times higher expression in different muscles as compared to the CMV promoter and also outperformed the Sk-CRM4/SPc5-12 and SPc5-12 promoters. Therefore, the computationally designed Sk-CRM4/Des chimeric promoter demonstrated improved muscle-specific performance as compared to the other promoters commonly used for muscle gene therapy.

In certain embodiments, the transcription cassette(s) further encodes a 5′-UTR region, a 3′-UTR region, and/or a polyA signal operably linked to the polynucleotide of the invention.

In certain embodiments, the 5′-UTR region comprises an intron. Expression of the target gene such as E-insulin can be enhanced due to the presence of an intron in the vector. The intron is typically positioned between the promoter and the coding region. While not wishing to be bound by any particular theory, the presence of the intron is believed to increase RNA stability in the nucleus due to the incorporation of mRNA into the spliceosome and promotes efficient export of spliced mRNA from the nucleus to the cytoplasm.

In certain embodiments, the introns contains regulatory sequences that affect tissue specificity and the expression level of the target gene. In certain embodiments, the intron is from the Ckm gene. In certain embodiments, the intron is the MVM intron.

In certain embodiments, the 3′-UTR comprises a (600-bp) post-transcriptional regulatory element of the woodchuck hepatitis virus (WPRE) which can lead to an enhancement of transgene expression in muscles. WPRE is believed to promote mRNA export from the nucleus and prevents post-translational gene silencing.

In certain embodiments, the AAV vector of the invention further comprises an miRNA detargeting sequence that suppresses expression of the target gene in a non-target tissue, such as liver or neuronal tissues. For this purpose, the binding sites for certain microRNA that are present only in the non-target organs are added to the 3′-UTR of the expression cassette (e.g., miRNA detargeting sites). If transgenic mRNA is expressed in a non-target organ, microRNA binds to such complementary detargeting sites on the transgene and initiates its degradation.

In certain embodiments, the vector is an AAV (adeno-associated viral) vector further comprising a 5′ ITR and/or a 3′ ITR.

As used herein, the term “AAV” is a standard abbreviation for adeno-associated virus. Adeno-associated virus is a single-stranded DNA parvovirus that grows only in cells in which certain functions are provided by a co-infecting helper virus. Numerous serotypes of AAV have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York, incorporated herein by reference). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” or “AAV vector genome (vg)” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

Another aspect of the invention provides an AAV viral particle comprising the AAV vector of the invention, encapsidated within a capsid.

An “AAV virion” or “AAV viral particle” refers to a viral particle composed of at least one AAV capsid protein and an encapsidated polynucleotide AAV vector. The particle comprises a heterologous polynucleotide (i.e., a polynucleotide other than a wild-type AAV genome such as a transgene to be delivered to a mammalian cell). Thus, production of AAV vector particle necessarily includes production of AAV vector, as such a vector is contained within an AAV vector particle.

In certain embodiments, the capsid has a serotype with muscle tropism, such as tropism for skeletal muscle, smooth muscle, or cardiac muscle.

In certain embodiments, the capsid has the serotype of AAV1, AAV2, AAV6, AAV7, AAV8, AAV9, AAV-DJ/8, AAV-Rh10, AAV-retro, AAV-PHP.B, AAV-PHP.eB, or AAV-PHP.S, or a genetically engineered AAV Capsid.

Production of pseudotyped rAAV is disclosed in, for example, WO 01/83692 (incorporated herein by reference). Other types of rAAV variants, for example rAAV with capsid mutations, are also contemplated. See, for example, Marsic et al., Molecular Therapy 22(11): 1900-1909, 2014 (incorporated herein by reference).

Another aspect of the invention provides a method of delivering an engineered insulin polypeptide to a muscle cell, the method comprising contacting the muscle cell with the AAV viral particle of the invention.

In certain embodiments, the muscle cell is in an animal, such as a human or a non-human mammal.

In certain embodiments, the animal has Type 1 or Type 2 diabetes, or hyperglycemia.

Another aspect of the invention provides a method of treating a subject in need of treatment for a disease treatable by insulin, the method comprising administering to the subject a therapeutically effective amount of the AAV viral particle of the invention.

In certain embodiments, the AAV viral particle is administered systemically, such as parental administration by injection, infusion or implantation.

In certain embodiments, the AAV viral particle is administered by intramuscular injection or intravenous injection.

In certain embodiments, the AAV viral particle is delivered intramuscularly.

In certain embodiments, the subject has Type 1 diabetes.

In certain embodiments, the subject has Type 2 diabetes.

In certain embodiments, the subject has hyperglycemia.

It should be understood that any one embodiment of any one of the aspects of the invention above, including specific embodiments described only in one section of the application, can be combined with one or more additional embodiments.

Another aspect of the invention provides a composition, such as a pharmaceutical composition, comprising the E-insulin polypeptide of the invention, the polynucleotide encoding the E-insulin polypeptide of the invention, the vector of the invention comprising the polynucleotide of the invention, the AAV vector genome of the invention comprising the polynucleotide of the invention, and/or the AAV viral particle of the invention encapsidating the AAV vector genome of the invention in a suitable capsid, such as a capsid with muscle tropism.

In certain embodiments, the composition of the invention is formulated for intramuscular injection or intravenous injection. In certain embodiments, the composition of the invention is formulated for systemic administration, such as parental administration by injection, infusion or implantation.

In certain embodiments, in any of the provided formulations or compositions, the buffer agent comprises one or more of tris, tricine, Bis-tricine, HEPES, MOPS, TES, TAPS, PIPES, and CAPS. For example, the buffer agent may comprise tris with pH 8.0 at concentration of about 5 mM to about 40 mM, or the buffer agent may comprise the tris with pH 8.0 at about 20 mM.

In certain embodiments, in any of the subject formulations or compositions, the ionic strength agent comprises one or more of potassium chloride (KCl), potassium acetate, potassium sulfate, ammonium sulfate, ammonium chloride (NH₄Cl), ammonium acetate, magnesium chloride (MgCl₂), magnesium acetate, magnesium sulfate, manganese chloride (MnCl₂), manganese acetate, manganese sulfate, sodium chloride (NaCl), sodium acetate, lithium chloride (LiCl), and lithium acetate.

For example, the ionic strength agent comprises MgCl₂ at a concentration of about 0.2 mM to about 4 mM; or the ionic strength agent comprises NaCl at a concentration of about 50 mM to about 500 mM; or the ionic strength agent comprises MgCl₂ at a concentration of about 0.2 mM to about 4 mM and NaCl at a concentration of about 50 mM to about 500 mM, or the ionic strength agent comprises MgCl₂ at a concentration of about 1 mM and NaCl at a concentration of about 200 mM.

In certain embodiments, in any of the provided formulations or compositions, the surfactant comprises one or more of a sulfonate, a sulfate, a phosphonate, a phosphate, a Poloxamer, and a cationic surfactant. For example, the Poloxamer comprises one or more of Poloxamer 124, Poloxamer 181, Poloxamer 184, Poloxamer 188, Poloxamer 237, Poloxamer 331, Poloxamer 338, and Poloxamer 407. The Poloxamer may be at a concentration of about 0.00001% to about 1%. An exemplary surfactant is Poloxamer 188 at a concentration of about 0.001%.

### 2. AAV

Recombinant AAV genomes of the invention comprise a polynucleotide or nucleic acid molecule of the invention, and one or more AAV ITRs flanking the polynucleotide or nucleic acid molecule. AAV DNA in the rAAV genomes may be from any AAV serotype for which a recombinant virus can be derived including, but not limited to, AAV serotypes AAV1, AAV2, AAV6, AAV7, AAV8, AAV9, AAV-DJ/8, AAV-Rh10, AAV-retro, AAV-PHP.B, AAV-PHP.eB, AAV-PHP.S, AAV-rh.10 or AAV-rh.74, or a genetically engineered AAV Capsid. In certain embodiments, the AAV viral particle has a serotype with muscle tropism, either exclusively, or preferentially.

Production of pseudotyped rAAV is disclosed in, for example, WO 01/83692. Other types of rAAV variants, for example rAAV with capsid mutations, are also contemplated. See, for example, Marsic et al., Molecular Therapy, 22(11): 1900-1909 (2014). The nucleotide sequences of the genomes of various AAV serotypes are known in the art.

In certain embodiments, to promote skeletal muscle specific expression, AAV-1, AAV-5, AAV-6, AAV-rh74, AAV-8 or AAV-9, for example, may be used.

In a related aspect, the invention also provides DNA plasmids comprising rAAV vector genomes of the invention. The DNA plasmids can be transferred to cells permissible for infection with a helper virus of AAV (e.g., adenovirus, El-deleted adenovirus or herpes virus) for assembly of the rAAV genome into infectious viral particles.

Techniques to produce rAAV particles, in which an AAV genome to be packaged, rep and cap genes, and helper virus functions are provided to a cell are standard in the art, and is commonly known as the so-called the “triple transfection” method, even though the method of introduction may not necessarily be the traditional means for plasmid transfection.

Production of rAAV generally requires that the following components be present within a single cell (denoted herein as a packaging cell): a rAAV genome, AAV rep and cap genes separate from (i.e., not in) the rAAV genome, and helper virus functions.

The AAV rep and cap genes may be from any AAV serotype for which recombinant virus can be derived and may be from a different AAV serotype than the rAAV genome ITRs, including, but not limited to, AAV serotypes AAV1, AAV2, AAV6, AAV7, AAV8, AAV9, AAV-DJ/8, AAV-Rh10, AAV-retro, AAV-PHP.B, AAV-PHP.eB, AAV-PHP.S, AAV-rh.10 or AAV-rh.74, or a genetically engineered AAV Capsid. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., Proc. Natl. Acad. S6. USA 79:2077-2081, 1982), addition of synthetic linkers containing restriction endonuclease cleavage sites (Laughlin et al., Gene, 23:65-73, 1983) or by direct, blunt-end ligation (Senapathy & Carter, J. Biol. Chem, 259:4661-4666, 1983). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); McLaughlin et al., J. Virol., 62:1963 (1988); and Lebkowski et al., 1988 Mol. Cell. Biol., 7:349 (1988). Samulski et al. (1989, J. Virol., 63:3822-3828); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. (1995) Vaccine 13:1244-1250; Paul et al. (1993) Human Gene Therapy 4:609-615; Clark et al. (1996) Gene Therapy 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with El of adenovirus), MRC-5 cells (human fetal fibroblasts), WT-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69427-443 (2002); U.S. Pat. No. 6,566,118 and WO 98/09657.

### 3. Composition and Pharmaceutical Composition

Another aspect of the invention provides compositions comprising the E-insulin of the invention, the polynucleotide encoding the E-insulin of the invention, and/or the vector comprising the polynucleotide of the invention.

In certain embodiments, the vector is an rAAV of the present invention.

Compositions described herein comprise rAAV in a pharmaceutically acceptable carrier. The compositions may also comprise other ingredients such as diluents and adjuvants.

Acceptable carriers, diluents and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Titers of rAAV to be administered in methods of the invention will vary depending, for example, on the particular rAAV, the mode of administration, the treatment goal, the individual, and the cell type(s) being targeted, and may be determined by methods standard in the art.

Exemplary titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml.

Dosages may also be expressed in units of viral genomes (vg). The titers of rAAV may be determined by the supercoiled plasmid quantitation standard or the linearized plasmid quantitation standard.

In certain embodiment, the disclosure provides methods of measuring the titer of an AAV vector, comprising tittering the AAV vector with PCR with a first primer and a second primer. In another embodiment, methods of measuring the titer of an AAV vector, comprising tittering the AAV vector with a probe.

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is T1DM, T2DM, or hyperglycemia.

Pharmaceutical compositions of the invention can be prepared as injectable formulations or as topical formulations to be delivered to the muscles by transdermal transport. Numerous formulations for both intramuscular injection and transdermal transport have been previously developed and can be used in the practice of the invention.

The rAAV can be used with any pharmaceutically acceptable carrier for ease of administration and handling. Thus, in another aspect, the application is directed to a formulation that comprises an rAAV that comprises a suitable AAV capsid with muscle tropism, such as AAV9, a buffer agent, an ionic strength agent, and a surfactant.

In one embodiment, the rAAV is at a concentration of 1.0×10¹² vg/mL to about 1.0×10¹⁶ vg/mL, or about 1.0×10¹² vg/mL to about 5.0×10¹⁴ vg/mL.

In another embodiment, the rAAV is at a concentration of about 5.0×10¹² vg/mL to about 1.0×10¹⁴ vg/mL based on a supercoiled plasmid as the quantitation standard.

In another embodiment, the rAAV is at a concentration of about 5.0×10¹² vg/mL to about 1.0×10¹⁴ vg/mL based on a linearized plasmid as the quantitation standard.

In another embodiment, the rAAV is at a concentration of about 2.0×10¹³ vg/mL based on a supercoiled plasmid as the quantitation standard.

In one embodiment, the concentration of rAAV in the composition or formulation is from 1.0×10¹³ vg/mL to 2.0×10¹⁴ vg/mL based on a supercoiled plasmid as the quantitation standard.

In another embodiment, the concentration is 2.0×10¹³ vg/mL, 4.0×10¹³ vg/mL, or 5.0×10¹³ vg/mL based on a supercoiled plasmid as the quantitation standard.

In one embodiment, the buffer agent comprises one or more of tris, tricine, Bis-tricine, HEPES, MOPS, TES, TAPS, PIPES, and CAPS.

In another embodiment, the buffer agent comprises tris with pH 8.0 at concentration of about 5 mM to about 40 M.

In one embodiment, the buffer agent comprises tris with pH 8.0 at about 20 mM.

In one embodiment, the ionic strength agent comprises one of more of potassium chloride (KCl), potassium acetate, potassium sulfate, ammonium sulfate, ammonium chloride (NH₄Cl), ammonium acetate, magnesium chloride (MgCl₂), magnesium acetate, magnesium sulfate, manganese chloride (MnCl₂), manganese acetate, manganese sulfate, sodium chloride (NaCl), sodium acetate, lithium chloride (LiCl), and lithium acetate.

In one embodiment, the ionic strength agent comprises MgCl₂ at a concentration of about 0.2 mM to about 4 mM. In another embodiment, the ionic strength agent comprises NaCl at a concentration of about 50 mM to about 500 mM. In another embodiment, the ionic strength agent comprises MgCl₂ at a concentration of about 0.2 mM to about 4 mM and NaCl at a concentration of about 50 mM to about 500 mM. In another embodiment, the ionic strength agent comprises MgCl₂ at a concentration of about 1 mM and NaCl at a concentration of about 200 mM.

In one embodiment, the surfactant comprises one or more of a sulfonate, a sulfate, a phosphonate, a phosphate, a Poloxamer, and a cationic surfactant.

In one embodiment, the Poloxamer comprises one or more of Poloxamer 124, Poloxamer 181, Poloxamer 184, Poloxamer 188, Poloxamer 237, Poloxamer 331, Poloxamer 338, and Poloxamer 407.

In one embodiment, the surfactant comprises the Poloxamer at a concentration of about 0.00001% to about 1%.

In another embodiment, the surfactant comprises Poloxamer 188 at a concentration of about 0.001%.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils.

Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, phenol, chlorobutanol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

### 4. Administration

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the E-insulin.

The invention provides for local administration and systemic administration of an effective dose of rAAV and compositions of the invention. For example, systemic administration is administration into the circulatory system so that the entire body is affected. Systemic administration includes enteral administration such as absorption through the gastrointestinal tract and parental administration through injection, infusion or implantation.

For example, administration of rAAV of the present invention may be accomplished by using any physical method that will transport the rAAV recombinant vector into the target tissue of an animal. Administration according to the invention includes, but is not limited to, injection into muscle, the bloodstream and/or directly into the liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained expression of E-insulin.

The present invention thus provides methods of administering/delivering rAAV which express E-insulin to a mammalian subject, preferably a human being. These methods include transducing tissues (including, but not limited to, tissues such as muscle, organs such as liver and brain, and glands such as salivary glands) with one or more rAAV of the present invention. Transduction may be carried out with gene cassettes comprising tissue specific control elements.

For example, one embodiment of the invention provides methods of transducing muscle cells and muscle tissues directed by muscle specific control elements, including, but not limited to, those derived from the actin and myosin gene families, such as from the myoD gene family [See Weintraub et al., Science, 251: 761-766 (1991)], the myocyte-specific enhancer binding factor MEF-2 [Cseijesi and Olson, Mol Cell Biol 11: 4854-4862 (1991)], control elements derived from the human skeletal actin gene [Muscat et al., Mol Cell Biol, 7: 4089-4099 (1987)], the cardiac actin gene, muscle creatine kinase sequence elements [See Johnson et al., Mol Cell Biol, 9:3393-3399 (1989)] and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA, 88: 5680-5684 (1991)), steroid-inducible elements and promoters including the glucocorticoid response element (GRE) (See Mader and White, Proc. Natl. Acad. Sci. USA 90: 5603-5607 (1993)), and other control elements.

Muscle tissue is an attractive target for in vivo DNA delivery, because it is not a vital organ and is easy to access. The invention contemplates sustained expression of E-insulin mRNAs from transduced myofibers.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of a polynucleotide of interest (e.g., a polynucleotide sequence encoding E-insulin) to a recipient cell either in vivo or in vitro, via a replication-deficient rAAV described resulting in expression of E-insulin by the recipient cell.

Thus, also described herein are methods of administering an effective dose (or doses, administered essentially simultaneously or doses given at intervals) of rAAV that encode E-insulin to a mammalian subject in need thereof.

### 5. Combination Therapy

Combination therapies are also contemplated by the invention. Combination as used herein includes both simultaneous treatment or sequential treatments. Combinations of methods of the invention with standard medical treatments are specifically contemplated, as are combinations with novel therapies.

In this regard, the combinations include administering to a subject one or more treatment suitable for treatment by insulin, before, simultaneous, or after the administration of an rAAV of the invention to a subject in need thereof.

In certain embodiments, the treatment suitable for treatment by insulin comprises medical nutrition therapy (MNT), a process by which the dietary plan of the subject or patient is tailored based on medical, lifestyle, and personal factors.

In certain embodiments, the treatment suitable for treatment by insulin comprises weight reduction treatment, e.g., for overweight (BMI ≥25 to 29.9 kg/m²) or obese (BMI ≥30 kg/m²) patients with type 2 diabetes. The weight reduction treatment may comprise reducing caloric intake, increasing physical activity, and/or behavior modification to achieve weight loss.

In certain embodiments, the treatment suitable for treatment by insulin comprises diet or diet control.

In certain embodiments, the treatment suitable for treatment by insulin comprises exercise, such as aerobic exercise that may include 30 to 60 minutes of moderate-intensity aerobic activity (40 to 60 percent VO₂ max) on most days of the week (e.g., at least 150 minutes of moderate-intensity aerobic exercise per week, spread over at least three days per week, with no more than two consecutive days without exercise); and/or resistance training (e.g., exercise with free weights or weight machines at least twice per week).

In certain embodiments, the treatment suitable for treatment by insulin comprises a pharmacological intervention that may comprise one or more of: metformin (e.g., 500 mg once daily with the evening meal and, if tolerated, add a second 500 mg dose with breakfast), GLP-1 receptor agonists (such as liraglutide, semaglutide, dulaglutide), SGLT2 inhibitor (such as empagliflozin, canagliflozin, and dapagliflozin), sulfonylurea (such as glipizide or glimepiride), DPP-4 inhibitor, repaglinide, or pioglitazone.

With the general parameters of the invention described above, additional aspects or features of the invention are provided in further details below.

All publications and patents mentioned herein are hereby incorporated by reference in their entirety as if each individual publication or patent was specifically and individually indicated to be incorporated by reference. In case of conflict, the present application, including any definitions herein, will control.

The invention is further described in the following Examples, which do not limit the scope of the invention described in the claims.

### EXAMPLES

### Example 1

One exemplary (non-limiting) engineered insulin (E-insulin) of the invention, including a signal peptide, was constructed, with the following polynucleotide and polypeptide sequences:

Engineered Insulin (E-Insulin) polynucleotide sequence:
(SEQ ID NO: 6)
ATGGACCCCCCACGCCCAGCATTGCTGGCCCTCCTTGCATTGCCCGCTTTGCTGTTGCTGCT
TCTTGCAGGTGCGCGGGCGTTTGTGAACCAACACCTGTGCGGCTCACACCTGGTGGAAGCTC
TCTACCTAGTGTGCGGGGAACGAGGCTTCTTCTACACACCCAAGACCCGCCGGAGAGGCGTG
TTCAGAAGAGAGGCAGAGGACCTGCAGGTGGGGCAGGTGGAGCTGGGCGGGGGCCCTGGTGC
AGGCAGCCTGCAGCCCTTGGCCCTGGAGGGGTCCCTGCAGAAGCGTCGGGGCGTCTTCCGCC
GCGGCATTGTGGAACAATGCTGTACCAGCATCTGCTCCCTCTACCAGCTGGAGAACTACTGC
AACTAG
Engineered Insulin (E-Insulin) polypeptide sequence:
(SEQ ID NO: 7)
MDPPRPALLALLALPALLLLLLAGARAFVNQHLCGSHLVEALYLVCGERGFFYTPKTRRRGV
FRREAEDLQVGQVELGGGPGAGSLQPLALEGSLQKRRGVFRRGIVEQCCTSICSLYQLENYC
N
Parts of the E-insulin sequence:
1.
Signal peptide:
(SEQ ID NO: 1)
MDPPRPALLALLALPALLLLLLAGARA
2.
B-chain:
(SEQ ID NO: 2)
FVNQHLCGSHLVEALYLVCGERGFFYTPKTRR
3.
1^{st} and 2^{nd} Heterologous Sequences:
(SEQ ID NO: 3)
RGVFRR
4.
C peptide:
(SEQ ID NO: 4)
EAEDLQVGQVELGGGPGAGSLQPLALEGSLQKR
5.
A-chain:
(SEQ ID NO: 5)
GIVEQCCTSICSLYQLENYCN

Note that the terminal one or two basic residue(s) in the B-chain and the C-peptide could be removed by enzymes such as carboxy peptidase after cleavage, and may or may not be present in the mature B-chain or C-peptide.

In this specific example, the sequence of RGVFRR (SEQ ID NO: 3) was inserted at the juncture of the natural human insulin B-chain and C-peptide as the first heterologous peptide, and again at the juncture of the natural human insulin C-peptide and A-chain as the second heterologous peptide, though it is not required for the two heterologous peptides to be the same.

For comparison, the full length human insulin polynucleotide and polypeptide sequences are provided below.

Full-length human insulin (Insulin-FL) polynucleotide sequence:
(SEQ ID NO: 8)
ATGGACCCCCCACGCCCAGCATTGCTGGCCCTCCTTGCATTGCCCGCTTTGCTGTTGCTGCT
TCTTGCAGGTGCGCGGGCGTTTGTGAACCAACACCTGTGCGGCTCACACCTGGTGGAAGCTC
TCTACCTAGTGTGCGGGGAACGAGGCTTCTTCTACACACCCAAGACCCGCCGGGAGGCAGAG
GACCTGCAGGTGGGGCAGGTGGAGCTGGGCGGGGGCCCTGGTGCAGGCAGCCTGCAGCCCTT
GGCCCTGGAGGGGTCCCTGCAGAAGCGTGGCATTGTGGAACAATGCTGTACCAGCATCTGCT
CCCTCTACCAGCTGGAGAACTACTGCAACTAG
Full-length human insulin (Insulin-FL) polypeptide sequence:
(SEQ ID NO: 9)
MDPPRPALLALLALPALLLLLLAGARAFVNQHLCGSHLVEALYLVCGERGFFYTPKTRREAE
DLQVGQVELGGGPGAGSLQPLALEGSLQKRGIVEQCCTSICSLYQLENYCN

The polynucleotide of SEQ ID NO: 6 was first inserted into an expression vector to show that E-insulin can be expressed in vitro.

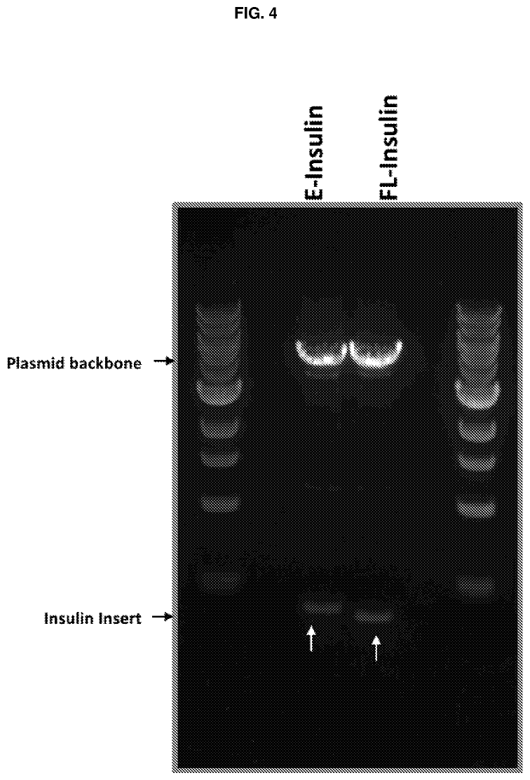

As shown in FIG. 4, upon restriction enzyme digestion, the E-insulin insert was released from the plasmid backbone, as were resolved on electrophoresis. A similarly digested control plasmid, with the wild-type full-length (FL) insulin coding sequence, released the slightly smaller FL insulin coding sequence fragment on the same gel.

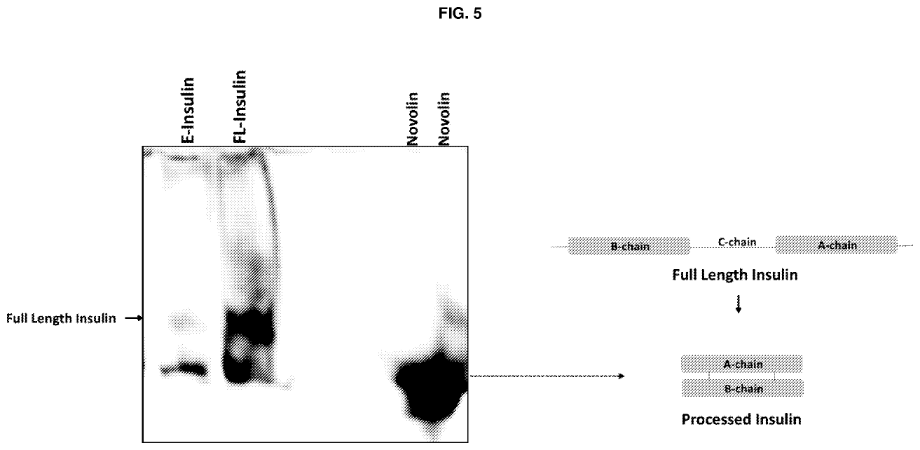

FIG. 5 shows that the E-insulin can be secreted and further be processed to mature forms, the same way the wild-type FL insulin can. Indeed, a majority of the E-insulin appeared to be processed to the mature form, which has a smaller molecular weight, and co-migrates with commercial insulin product Novolin. Meanwhile, FL insulin has a significant portion remain unprocessed to the mature form, and remains full length (including the C-peptide).

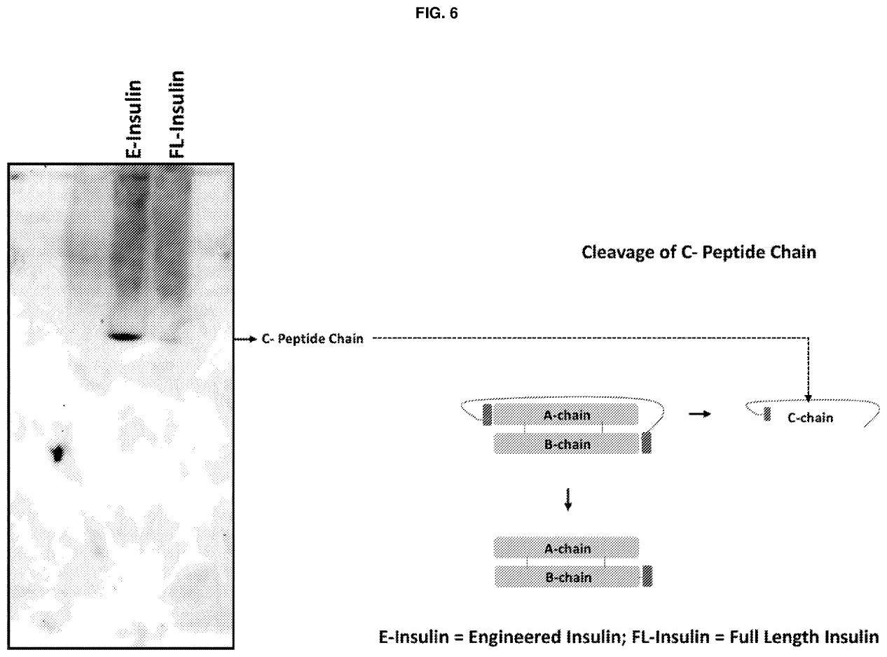

As a further evidence that the E-insulin was fully processed, Western blotting against the C-peptide, which was supposed to be released upon protease digestion to produce the mature insulin, showed that the C-peptide was indeed released in the E-insulin sample (FIG. 6), to a much greater extent than that for the wild-type full length (FL) insulin.

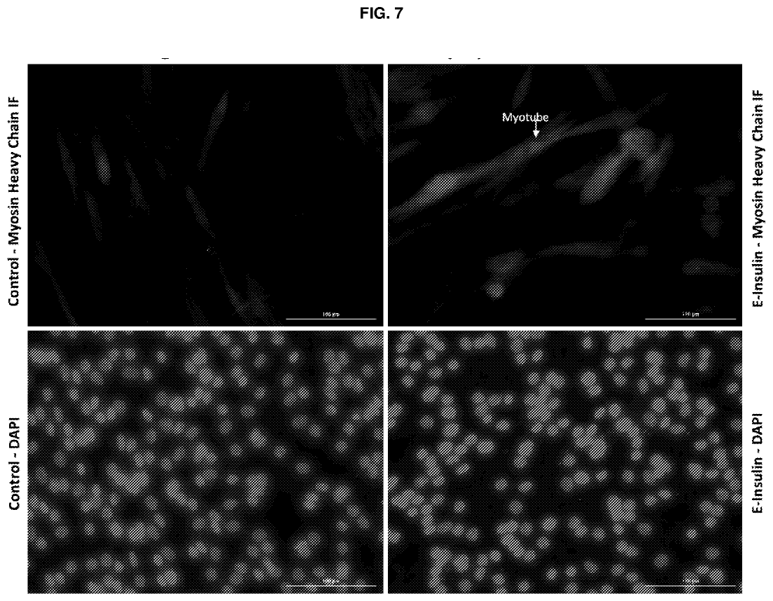

FIG. 7 shows that the secreted E-insulin was capable of being a potent inducer in muscle cells (myoblasts) by promoting their differentiation into myotubes. When the coding sequence of E-insulin was introduced into muscle cells, immunofluorescent (IF) staining of myosin heavy chain—a myotube differentiation marker—showed strong signal in the E-insulin panel (FIG. 7, upper right hand panel), while only weak signal was observed in the control FL insulin panel (FIG. 7, upper left hand panel), even though both samples contained equivalent amount of cells as evidenced by DAPI staining (FIG. 7, lower left and right panels).

The polynucleotide of SEQ ID NO: 6 is then inserted into an AAV viral vector, under the control of one of the promoters of the invention, such as a constitutively active eukaryotic promoter, a muscle-specific promoter (such as a CK8 promoter), and/or a synthetic promoter (such as CAG, CBA, or derivative thereof).

Such transcription unit may optionally further comprise a 5′ UTR region, a heterologous intron to enhance expression of the insulin, a 3′ UTR region, and/or a polyA tail.

The AAV vector genome encompassing such a transcription unit is flanked by at least one functional ITR, such as a 5′ ITR and/or a 3′ ITR from AAV2.

The AAV vector genome can be encapsidated in any AAV capsid with preferential tropism for muscle tissues, such as skeletal muscle, smooth muscle (e.g., diaphragm), and/or cardiac muscle. Exemplary suitable AAV capsids include AAV1, AAV2, AAV6, AAV7, AAV8, AAV9, AAV-DJ/8, AAV-Rh10, AAV-retro, AAV-PHP.B, AAV-PHP.eB, or AAV-PHP.S, or a genetically engineered AAV Capsid. For example, AAV9 capsid may be used.

AAV viral particles can be produced using any standard methods, such as the triple transfection method in a host cell (e.g., HEK293) that permits assembling of AAV viral particles from the AAV vector genome described above in one vector, AAV rep and cap genes on a second vector, and helper genes for AAV packaging in a 3^{rd} vector.

The harvested AAV viral particles can then be tittered and administered to an animal or subject in need thereof, such as one having Type 1 diabetes (T1DM), Type 2 diabetes (T2DM), and/or hyperglycemia. The injection can be intramuscular injection (i.m.).

The animal or subject receiving such AAV injection can produce the E-insulin of the invention, that can be secreted into the blood stream from muscle tissues expressing such E-insulin, thereby treating T1DM, T2DM, and/or hyperglycemia.

### Example 2

This example demonstrates intramuscular secretion of the subject human insulin from muscle tissue, and the effect of such muscle-secreted insulin in blood glucose reduction.

Specifically, diabetic mice were intramuscularly dosed with an AAV vector harboring a polynucleotide encoding an E-Insulin of the disclosure. Experimental diabetes was induced in 6-week old C57BL6/J mice to develop diabetes, by inducing with Streptozotocin (about 50 mg/kg) in 0.1 M Citrate buffer at pH4.5 daily IP injection for 5 consecutive days. After Streptozotocin treatment, mice were allowed to develop diabetes for two weeks until they reached fasting blood glucose ranging between 300-400 mg/dL.

An AAV9 vector harboring an E-insulin coding sequence was injected intramuscularly in such diabetic mice, with the AAV9 viral particles suspended in about 200 L of PBS buffer, which total volume was split between muscles of the two hind limbs of each experimental animal. Three AAV doses comprising about 2×10¹³ (2E13) vg/kg, about 2×10¹² (2E12) vg/kg, and about 2×10¹¹ (2E11) vg/kg were used for the study with controls.

After AAV dosing, fasting blood glucose was measured in all animals using a handheld glucometer on a weekly basis. Preliminary analysis of the gathered data showed reduction in fasting blood glucose levels in animals intramuscularly dosed with the AAV9 viral particles harboring the E-Insulin coding sequence (data not shown).

Further detailed analysis of human insulin secretion from muscle tissues into the bloodstream were being conducted.

1. An engineered insulin polypeptide (E-insulin) engineered from a wild-type insulin sequence in a mammal, comprising:
(a) a first cleavable heterologous peptide, between the insulin B-chain and the insulin C-peptide of the wild-type insulin; and,
(b) a second cleavable heterologous peptide, between the insulin C-peptide and the insulin A-chain of the wild-type insulin;
wherein the first and the second cleavable heterologous peptides are both native sequences of the mammal.

2. The E-insulin of claim 1, wherein the mammal is a human.

3. The E-insulin of claim 1 or 2, wherein the first and the second cleavable heterologous peptides are both cleavable by a serine endoprotease expressed in muscle tissues.

4. The E-insulin of any one of claims 1-3, wherein the serine endoprotease is a subtilisin-like endoprotease, such as furin, PACE4, PC4, PC5/6, or LPC/PC8.

5. The E-insulin of any one of claims 1-4, wherein the first cleavable heterologous peptide and the second cleavable heterologous peptide independently comprise a sequence motif Arg-Xaa-Xaa-Arg (wherein Xaa is any amino acid) or Lys/Arg-Arg.

6. The E-insulin of any one of claims 3-5, wherein the serine endoprotease is present or expressed in trans-Golgi network (TGN), such as TGN of muscle tissue/cell.

7. The E-insulin of any one of claims 3-6, wherein the serine endoprotease is Furin.

8. The E-insulin of any one of claims 1-7, wherein the first cleavable heterologous peptide and the second cleavable heterologous peptide both comprise, consist essentially of, or consist of an amino acid sequence independently selected from
(SEQ ID NO: 3)
RGVFRR;
RAKR; RKKR; RKYR; RRKR RRRR; RQRR; LSRR;
RKRR; RHKR; RERR; RPRR; RARR; RSAR; LRKR; RTRR RTAR; RNTR; RLRR;
RWRR; RGKR; RVKR; RHPR; RRGR; RVRR; RDRR; RTGR REAR; RFKR;
RYKR; RHAR; RLLR; RVGR; RRAR; HPKR; RFPR; RRTR; RNQR; RSRR
RRRK; RSIR; RTPR; RPAR; RNHR; RKTR; RKNR; RLKR; RNRR; RKSR;
RMKR; RNKR RQQR; RIRR; REIR; REKR; RMRR; RPDR; RIVR; RPKR; RLKK;
RSKR; RVAR; RGPR RVCR; RAPR; RQIR; RYRR; RKIR; RMIR; RVRK; RRVR;
RCQR; RCKR; VRKR; RPVR RHTR; RCRR; RGRR; RISR; RAAR; RLGR; RYPR;
RAGR; RIIR; KSAR; VSRR; RQCR RVKK; RIGR; RTTR; RHRR; VVAR; RSHR;
RYSR; RCIR; RCYR; RFRK; RYFR; RTMR RHGR; RSPR; RETR; RKFR; RHLR;
RKGR; RRKK; RWKR; RVLR; RIKR; VKKR; RFRR RALR; MKKR; RVTR;
NCSR; KRKR; RSSR; RMAR; KRRR; RRPR; TIKR; HLKR; RFER RASR; RRSR;
VRRR; KAKR; RDKR; KVKR; RFLR; RLPR; RPPR; RHSR; RSGR; RICR HKKR;
RSLR; RLVR; RVPR; RFSR; RTIR; RLTR; RKAR; RAQR; RQMR; RFVR RSNR;
RQPR; CVRR; RSYR; IKKR; RHVR; RLAR; RPLR; IGKR; VTKR; RRHR; RRNR
VHKR; RCPR; RWPR; SKER; RIKK; RIFR; RVWR; LEGR; LKKR; VFRR; IRKR.

9. The E-insulin of any one of claims 1-8, wherein the insulin B-chain comprises, consists essentially of, or consists of the amino acid sequence of SEQ ID NO: 2.

10. The E-insulin of any one of claims 1-9, wherein the insulin C-peptide comprises, consists essentially of, or consists of the amino acid sequence of SEQ ID NO: 4.

11. The E-insulin of any one of claims 1-10, wherein the insulin A-chain comprises, consists essentially of, or consists of the amino acid sequence of SEQ ID NO: 5.

12. The E-insulin of any one of claims 1-11, further comprising a signal peptide.

13. The E-insulin of claim 12, wherein the signal peptide comprises, consists essentially of, or consists of the amino acid sequence of SEQ ID NO: 1.

14. The E-insulin of any one of claims 1-13, wherein the insulin A-chain and the insulin B-chain are covalently linked by a disulfide bond.

15. The E-insulin of any one of claims 1-14, wherein the insulin A-chain and the insulin B-chain are covalently linked by two disulfide bonds.

16. The E-insulin of any one of claims 1-15, further comprising an H10D substitution on the insulin B-chain.

17. A mature E-insulin resulting from cleavage of the E-insulin of any one of claims 1-16 at said first cleavable heterologous peptide and said second cleavable heterologous peptide, wherein said insulin A-chain and said insulin B-chain are covalently linked by two disulfide bonds.

18. The mature E-insulin of claim 17, comprising a first remnant sequence of the first cleavable heterologous peptide, optionally, the first remnant sequence (and further optionally the last one or two basic residue(s) at the C-terminus of SEQ ID NO: 2) is removed by a carboxypeptidase (such as carboxypeptidase E) in said mature E-insulin.

19. A cleaved insulin C-peptide resulting from cleavage of the E-insulin of any one of claims 1-16 at said first cleavable heterologous peptide and said second cleavable heterologous peptide, wherein said cleaved insulin C-peptide comprises a remnant sequence of the second cleavable heterologous peptide; optionally, the second remnant sequence (and further optionally the last one or two basic residue(s) at the C-terminus of SEQ ID NO: 4) is removed by a carboxypeptidase (such as carboxypeptidase E) in said cleaved insulin C-peptide.

20. A polynucleotide encoding the E-insulin of any one of claims 1-16.

21. A vector comprising the polynucleotide of claim 20.

22. The vector of claim 21, comprising a promoter operably linked to a transcription cassette comprising the polynucleotide of claim 20.

23. The vector of claim 22, wherein the promoter comprises:
(1) a constitutively active eukaryotic promoter,
(2) a muscle-specific promoter, and/or
(3) a synthetic promoter.

24. The vector of claim 22 or 23, comprising two promoters.

25. The vector of any one of claims 21-24, comprising:
(1) a first transcription cassette comprising the polynucleotide of claim 16 operably linked to a first promoter (e.g., a muscle-specific promoter such as MCK, or a constitutive promoter such as CBA or CAG); and,
(2) a second transcription cassette comprising the polynucleotide of claim 16 operably linked to a second promoter, such as a synthetic promoter;
wherein the polynucleotides of claim 20 in the first and the second transcription cassettes are independently selected from the E-insulin of any one of claims 1-16.

26. The vector of any one of claims 22-25, wherein the transcription cassette(s) further encodes a 5′-UTR region, a 3′-UTR region, and/or a polyA signal operably linked to the polynucleotide of claim 20.

27. The vector of any one of claims 21-26, which is an AAV (adeno-associated viral) vector further comprising a 5′ ITR and/or a 3′ ITR.

28. An AAV viral particle comprising the AAV vector of claim 27, encapsidated within a capsid.

29. The AAV viral particle of claim 28, wherein the capsid has a serotype with muscle tropism, such as tropism for skeletal muscle, smooth muscle, or cardiac muscle.

30. The AAV viral particle of claim 28 or 29, wherein the capsid has the serotype of AAV1, AAV2, AAV6, AAV7, AAV8, AAV9, AAV-DJ/8, AAV-Rh10, AAV-retro, AAV-PHP.B, AAV-PHP.eB, or AAV-PHP.S, or a genetically engineered AAV Capsid.

31. A method of delivering an engineered insulin polypeptide to a muscle cell, the method comprising contacting the muscle cell with the AAV viral particle of claim 29 or 30.

32. The method of claim 31, wherein the muscle cell is in an animal, such as a human or a non-human mammal.

33. The method of claim 32, wherein animal has Type 1 or Type 2 diabetes, or hyperglycemia.

34. A method of treating a subject in need of treatment for a disease treatable by insulin, the method comprising administering to the subject a therapeutically effective amount of the AAV viral particle of any one of claims 27-30.

35. The method of claim 34, wherein the AAV viral particle is delivered intramuscularly.

36. The method of claim 34 or 35, wherein the subject has Type 1 diabetes, Type 2 diabetes, or hyperglycemia.

37. The method of any one of claims 34-36, wherein the subject has hyperglycemia.


SEQUENCE LISTING (170 sequences)

SEQ ID NO: 1 - protein, 27 aa, synthetic construct
  REGION 1..27: note = Synthetic: Signal peptide
MDPPRPALLALLALPALLLLLLAGARA

SEQ ID NO: 2 - protein, 32 aa, synthetic construct
  REGION 1..32: note = Synthetic: B-chain
FVNQHLCGSHLVEALYLVCGERGFFYTPKTRR

SEQ ID NO: 3 - protein, 6 aa, synthetic construct
  REGION 1..6: note = Synthetic: 1st and 2nd Heterologous Sequences
RGVFRR

SEQ ID NO: 4 - protein, 33 aa, synthetic construct
  REGION 1..33: note = Synthetic: C peptide
EAEDLQVGQVELGGGPGAGSLQPLALEGSLQKR

SEQ ID NO: 5 - protein, 21 aa, synthetic construct
  REGION 1..21: note = Synthetic: A-chain
GIVEQCCTSICSLYQLENYCN

SEQ ID NO: 6 - DNA, 378 bp, synthetic construct
  misc_feature 1..378: note = Synthetic: Engineered Insulin (E-Insulin) polynucleotide sequence
  source 1..378: mol_type = other DNA
atggaccccccacgcccagcattgctggccctccttgcattgcccgctttgctgttgctgcttcttgcaggtgcgcgggcgtttgtgaaccaacacctgtgcggctcacacctggtggaagctctctacctagtgtgcggggaacgaggcttcttctacacacccaagacccgccggagaggcgtgttcagaagagaggcagaggacctgcaggtggggcaggtggagctgggcgggggccctggtgcaggcagcctgcagcccttggccctggaggggtccctgcagaagcgtcggggcgtcttccgccgcggcattgtggaacaatgctgtaccagcatctgctccctctaccagctggagaactactgcaactag

SEQ ID NO: 7 - protein, 125 aa, synthetic construct
  REGION 1..125: note = Synthetic: Engineered Insulin (E-Insulin) polypeptide sequence
MDPPRPALLALLALPALLLLLLAGARAFVNQHLCGSHLVEALYLVCGERGFFYTPKTRRRGVFRREAEDLQVGQVELGGGPGAGSLQPLALEGSLQKRRGVFRRGIVEQCCTSICSLYQLENYCN

SEQ ID NO: 8 - DNA, 342 bp, Homo sapiens
  misc_feature 1..342: note = Full-length human insulin (Insulin-FL) polynucleotide sequence
  source 1..342: mol_type = genomic DNA
atggaccccccacgcccagcattgctggccctccttgcattgcccgctttgctgttgctgcttcttgcaggtgcgcgggcgtttgtgaaccaacacctgtgcggctcacacctggtggaagctctctacctagtgtgcggggaacgaggcttcttctacacacccaagacccgccgggaggcagaggacctgcaggtggggcaggtggagctgggcgggggccctggtgcaggcagcctgcagcccttggccctggaggggtccctgcagaagcgtggcattgtggaacaatgctgtaccagcatctgctccctctaccagctggagaactactgcaactag

SEQ ID NO: 9 - protein, 113 aa, Homo sapiens
  REGION 1..113: note = misc_feature - Full-length human insulin (Insulin - FL) polypeptide sequence
MDPPRPALLALLALPALLLLLLAGARAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 10 - protein, 17 aa, synthetic construct
  REGION 1..17: note = Synthetic: C peptide
GAGSLQPLALEGSLQKR

SEQ ID NO: 11 - protein, 4 aa, synthetic construct
  REGION 1..4: note = Synthetic
RAKR

SEQ ID NO: 12 - protein, 4 aa, synthetic construct
  REGION 1..4: note = Synthetic
RKKR

SEQ ID NO: 13 - protein, 4 aa, synthetic construct
  REGION 1..4: note = Synthetic
RKYR

SEQ ID NO: 14 - protein, 8 aa, synthetic construct
  REGION 1..8: note = Synthetic
RRKRRRRR

SEQ ID NO: 15 - protein, 4 aa, synthetic construct
  REGION 1..4: note = Synthetic
RQRR

SEQ ID NO: 16 - protein, 4 aa, synthetic construct
  REGION 1..4: note = Synthetic
LSRR

SEQ ID NO: 17 - protein, 4 aa, synthetic construct
  REGION 1..4: note = Synthetic
RKRR

SEQ ID NO: 18 - protein, 4 aa, synthetic construct
  REGION 1..4: note = Synthetic
RHKR

SEQ ID NO: 19 - protein, 4 aa, synthetic construct
  REGION 1..4: note = Synthetic
RERR

SEQ ID NO: 20 - protein, 4 aa, synthetic construct
  REGION 1..4: note = Synthetic
RPRR

SEQ ID NO: 21 - protein, 4 aa, synthetic construct
  REGION 1..4: note = Synthetic
RARR

SEQ ID NO: 22 - protein, 4 aa, synthetic construct
  REGION 1..4: note = Synthetic
RSAR

SEQ ID NO: 23 - protein, 4 aa, synthetic construct
  REGION 1..4: note = Synthetic
LRKR

SEQ ID NO: 24 - protein, 8 aa, synthetic construct
  REGION 1..8: note = Synthetic
RTRRRTAR

SEQ ID NO: 25 - protein, 4 aa, synthetic construct
  REGION 1..4: note = Synthetic
RNTR

SEQ ID NO: 26 - protein, 4 aa, synthetic construct
  REGION 1..4: note = Synthetic
RLRR

SEQ ID NO: 27 - protein, 4 aa, synthetic construct
  REGION 1..4: note = Synthetic
RWRR

SEQ ID NO: 28 - protein, 4 aa, synthetic construct
  REGION 1..4: note = Synthetic
RGKR

SEQ ID NO: 29 - protein, 4 aa, synthetic construct
  REGION 1..4: note = Synthetic
RVKR

SEQ ID NO: 30 - protein, 4 aa, synthetic construct
  REGION 1..4: note = Synthetic
RHPR

SEQ ID NO: 31 - protein, 4 aa, synthetic construct
  REGION 1..4: note = Synthetic
RRGR

SEQ ID NO: 32 - protein, 4 aa, synthetic construct
  REGION 1..4: note = Synthetic
RVRR

SEQ ID NO: 33 - protein, 4 aa, synthetic construct
  REGION 1..4: note = Synthetic
RDRR

SEQ ID NO: 34 - protein, 8 aa, synthetic construct
  REGION 1..8: note = Synthetic
RTGRREAR

SEQ ID NO: 35 - protein, 4 aa, synthetic construct
  REGION 1..4: note = Synthetic
RFKR

SEQ ID NO: 36 - protein, 4 aa, synthetic construct
  REGION 1..4: note = Synthetic
RYKR

SEQ ID NO: 37 - protein, 4 aa, synthetic construct
  REGION 1..4: note = Synthetic
RHAR

SEQ ID NO: 38 - protein, 4 aa, synthetic construct
  REGION 1..4: note = Synthetic
RLLR

SEQ ID NO: 39 - protein, 4 aa, synthetic construct
  REGION 1..4: note = Synthetic
RVGR

SEQ ID NO: 40 - protein, 4 aa, synthetic construct
  REGION 1..4: note = Synthetic
RRAR

SEQ ID NO: 41 - protein, 4 aa, synthetic construct
  REGION 1..4: note = Synthetic
HPKR

SEQ ID NO: 42 - protein, 4 aa, synthetic construct
  REGION 1..4: note = Synthetic
RFPR

SEQ ID NO: 43 - protein, 4 aa, synthetic construct
  REGION 1..4: note = Synthetic
RRTR

SEQ ID NO: 44 - protein, 4 aa, synthetic construct
  REGION 1..4: note = Synthetic
RNQR

SEQ ID NO: 45 - protein, 8 aa, synthetic construct
  REGION 1..8: note = Synthetic
RSRRRRRK

SEQ ID NO: 46 - protein, 4 aa, synthetic construct
  REGION 1..4: note = Synthetic
RSIR

SEQ ID NO: 47 - protein, 4 aa, synthetic construct
  REGION 1..4: note = Synthetic
RTPR

SEQ ID NO: 48 - protein, 4 aa, synthetic construct
  REGION 1..4: note = Synthetic
RPAR

SEQ ID NO: 49 - protein, 4 aa, synthetic construct
  REGION 1..4: note = Synthetic
RNHR

SEQ ID NO: 50 - protein, 4 aa, synthetic construct
  REGION 1..4: note = Synthetic
RKTR

SEQ ID NO: 51 - protein, 4 aa, synthetic construct
  REGION 1..4: note = Synthetic
RKNR

SEQ ID NO: 52 - protein, 4 aa, synthetic construct
  REGION 1..4: note = Synthetic
RLKR

SEQ ID NO: 53 - protein, 4 aa, synthetic construct
  REGION 1..4: note = Synthetic
RNRR

SEQ ID NO: 54 - protein, 4 aa, synthetic construct
  REGION 1..4: note = Synthetic
RKSR

SEQ ID NO: 55 - protein, 4 aa, synthetic construct
  REGION 1..4: note = Synthetic
RMKR

SEQ ID NO: 56 - protein, 8 aa, synthetic construct
  REGION 1..8: note = Synthetic
RNKRRQQR

SEQ ID NO: 57 - protein, 4 aa, synthetic construct
  REGION 1..4: note = Synthetic
RIRR

SEQ ID NO: 58 - protein, 4 aa, synthetic construct
  REGION 1..4: note = Synthetic
REIR

SEQ ID NO: 59 - protein, 4 aa, synthetic construct
  REGION 1..4: note = Synthetic
REKR

SEQ ID NO: 60 - protein, 4 aa, synthetic construct
  REGION 1..4: note = Synthetic
RMRR

SEQ ID NO: 61 - protein, 4 aa, synthetic construct
  REGION 1..4: note = Synthetic
RPDR

SEQ ID NO: 62 - protein, 4 aa, synthetic construct
  REGION 1..4: note = Synthetic
RIVR

SEQ ID NO: 63 - protein, 4 aa, synthetic construct
  REGION 1..4: note = Synthetic
RPKR

SEQ ID NO: 64 - protein, 4 aa, synthetic construct
  REGION 1..4: note = Synthetic
RLKK

SEQ ID NO: 65 - protein, 4 aa, synthetic construct
  REGION 1..4: note = Synthetic
RSKR

SEQ ID NO: 66 - protein, 4 aa, synthetic construct
  REGION 1..4: note = Synthetic
RVAR

SEQ ID NO: 67 - protein, 8 aa, synthetic construct
  REGION 1..8: note = Synthetic
RGPRRVCR

SEQ ID NO: 68 - protein, 4 aa, synthetic construct
  REGION 1..4: note = Synthetic
RAPR

SEQ ID NO: 69 - protein, 4 aa, synthetic construct
  REGION 1..4: note = Synthetic
RQIR

SEQ ID NO: 70 - protein, 4 aa, synthetic construct
  REGION 1..4: note = Synthetic
RYRR

SEQ ID NO: 71 - protein, 4 aa, synthetic construct
  REGION 1..4: note = Synthetic
RKIR

SEQ ID NO: 72 - protein, 4 aa, synthetic construct
  REGION 1..4: note = Synthetic
RMIR

SEQ ID NO: 73 - protein, 4 aa, synthetic construct
  REGION 1..4: note = Synthetic
RVRK

SEQ ID NO: 74 - protein, 4 aa, synthetic construct
  REGION 1..4: note = Synthetic
RRVR

SEQ ID NO: 75 - protein, 4 aa, synthetic construct
  REGION 1..4: note = Synthetic
RCQR

SEQ ID NO: 76 - protein, 4 aa, synthetic construct
  REGION 1..4: note = Synthetic
RCKR

SEQ ID NO: 77 - protein, 4 aa, synthetic construct
  REGION 1..4: note = Synthetic
VRKR

SEQ ID NO: 78 - protein, 8 aa, synthetic construct
  REGION 1..8: note = Synthetic
RPVRRHTR

SEQ ID NO: 79 - protein, 4 aa, synthetic construct
  REGION 1..4: note = Synthetic
RCRR

SEQ ID NO: 80 - protein, 4 aa, synthetic construct
  REGION 1..4: note = Synthetic
RGRR

SEQ ID NO: 81 - protein, 4 aa, synthetic construct
  REGION 1..4: note = Synthetic
RISR

SEQ ID NO: 82 - protein, 4 aa, synthetic construct
  REGION 1..4: note = Synthetic
RAAR

SEQ ID NO: 83 - protein, 4 aa, synthetic construct
  REGION 1..4: note = Synthetic
RLGR

SEQ ID NO: 84 - protein, 4 aa, synthetic construct
  REGION 1..4: note = Synthetic
RYPR

SEQ ID NO: 85 - protein, 4 aa, synthetic construct
  REGION 1..4: note = Synthetic
RAGR

SEQ ID NO: 86 - protein, 4 aa, synthetic construct
  REGION 1..4: note = Synthetic
RIIR

SEQ ID NO: 87 - protein, 4 aa, synthetic construct
  REGION 1..4: note = Synthetic
KSAR

SEQ ID NO: 88 - protein, 4 aa, synthetic construct
  REGION 1..4: note = Synthetic
VSRR

SEQ ID NO: 89 - protein, 8 aa, synthetic construct
  REGION 1..8: note = Synthetic
RQCRRVKK

SEQ ID NO: 90 - protein, 4 aa, synthetic construct
  REGION 1..4: note = Synthetic
RIGR

SEQ ID NO: 91 - protein, 4 aa, synthetic construct
  REGION 1..4: note = Synthetic
RTTR

SEQ ID NO: 92 - protein, 4 aa, synthetic construct
  REGION 1..4: note = Synthetic
RHRR

SEQ ID NO: 93 - protein, 4 aa, synthetic construct
  REGION 1..4: note = Synthetic
VVAR

SEQ ID NO: 94 - protein, 4 aa, synthetic construct
  REGION 1..4: note = Synthetic
RSHR

SEQ ID NO: 95 - protein, 4 aa, synthetic construct
  REGION 1..4: note = Synthetic
RYSR

SEQ ID NO: 96 - protein, 4 aa, synthetic construct
  REGION 1..4: note = Synthetic
RCIR

SEQ ID NO: 97 - protein, 4 aa, synthetic construct
  REGION 1..4: note = Synthetic
RCYR

SEQ ID NO: 98 - protein, 4 aa, synthetic construct
  REGION 1..4: note = Synthetic
RFRK

SEQ ID NO: 99 - protein, 4 aa, synthetic construct
  REGION 1..4: note = Synthetic
RYFR

SEQ ID NO: 100 - protein, 8 aa, synthetic construct
  REGION 1..8: note = Synthetic
RTMRRHGR

SEQ ID NO: 101 - protein, 4 aa, synthetic construct
  REGION 1..4: note = Synthetic
RSPR

SEQ ID NO: 102 - protein, 4 aa, synthetic construct
  REGION 1..4: note = Synthetic
RETR

SEQ ID NO: 103 - protein, 4 aa, synthetic construct
  REGION 1..4: note = Synthetic
RKFR

SEQ ID NO: 104 - protein, 4 aa, synthetic construct
  REGION 1..4: note = Synthetic
RHLR

SEQ ID NO: 105 - protein, 4 aa, synthetic construct
  REGION 1..4: note = Synthetic
RKGR

SEQ ID NO: 106 - protein, 4 aa, synthetic construct
  REGION 1..4: note = Synthetic
RRKK

SEQ ID NO: 107 - protein, 4 aa, synthetic construct
  REGION 1..4: note = Synthetic
RWKR

SEQ ID NO: 108 - protein, 4 aa, synthetic construct
  REGION 1..4: note = Synthetic
RVLR

SEQ ID NO: 109 - protein, 4 aa, synthetic construct
  REGION 1..4: note = Synthetic
RIKR

SEQ ID NO: 110 - protein, 4 aa, synthetic construct
  REGION 1..4: note = Synthetic
VKKR

SEQ ID NO: 111 - protein, 8 aa, synthetic construct
  REGION 1..8: note = Synthetic
RFRRRALR

SEQ ID NO: 112 - protein, 4 aa, synthetic construct
  REGION 1..4: note = Synthetic
MKKR

SEQ ID NO: 113 - protein, 4 aa, synthetic construct
  REGION 1..4: note = Synthetic
RVTR

SEQ ID NO: 114 - protein, 4 aa, synthetic construct
  REGION 1..4: note = Synthetic
NCSR

SEQ ID NO: 115 - protein, 4 aa, synthetic construct
  REGION 1..4: note = Synthetic
KRKR

SEQ ID NO: 116 - protein, 4 aa, synthetic construct
  REGION 1..4: note = Synthetic
RSSR

SEQ ID NO: 117 - protein, 4 aa, synthetic construct
  REGION 1..4: note = Synthetic
RMAR

SEQ ID NO: 118 - protein, 4 aa, synthetic construct
  REGION 1..4: note = Synthetic
KRRR

SEQ ID NO: 119 - protein, 4 aa, synthetic construct
  REGION 1..4: note = Synthetic
RRPR

SEQ ID NO: 120 - protein, 4 aa, synthetic construct
  REGION 1..4: note = Synthetic
TIKR

SEQ ID NO: 121 - protein, 4 aa, synthetic construct
  REGION 1..4: note = Synthetic
HLKR

SEQ ID NO: 122 - protein, 8 aa, synthetic construct
  REGION 1..8: note = Synthetic
RFERRASR

SEQ ID NO: 123 - protein, 4 aa, synthetic construct
  REGION 1..4: note = Synthetic
RRSR

SEQ ID NO: 124 - protein, 4 aa, synthetic construct
  REGION 1..4: note = Synthetic
VRRR

SEQ ID NO: 125 - protein, 4 aa, synthetic construct
  REGION 1..4: note = Synthetic
KAKR

SEQ ID NO: 126 - protein, 4 aa, synthetic construct
  REGION 1..4: note = Synthetic
RDKR

SEQ ID NO: 127 - protein, 4 aa, synthetic construct
  REGION 1..4: note = Synthetic
KVKR

SEQ ID NO: 128 - protein, 4 aa, synthetic construct
  REGION 1..4: note = Synthetic
RFLR

SEQ ID NO: 129 - protein, 4 aa, synthetic construct
  REGION 1..4: note = Synthetic
RLPR

SEQ ID NO: 130 - protein, 4 aa, synthetic construct
  REGION 1..4: note = Synthetic
RPPR

SEQ ID NO: 131 - protein, 4 aa, synthetic construct
  REGION 1..4: note = Synthetic
RHSR

SEQ ID NO: 132 - protein, 4 aa, synthetic construct
  REGION 1..4: note = Synthetic
RSGR

SEQ ID NO: 133 - protein, 8 aa, synthetic construct
  REGION 1..8: note = Synthetic
RICRHKKR

SEQ ID NO: 134 - protein, 4 aa, synthetic construct
  REGION 1..4: note = Synthetic
RSLR

SEQ ID NO: 135 - protein, 4 aa, synthetic construct
  REGION 1..4: note = Synthetic
RLVR

SEQ ID NO: 136 - protein, 4 aa, synthetic construct
  REGION 1..4: note = Synthetic
RVPR

SEQ ID NO: 137 - protein, 4 aa, synthetic construct
  REGION 1..4: note = Synthetic
RFSR

SEQ ID NO: 138 - protein, 4 aa, synthetic construct
  REGION 1..4: note = Synthetic
RTIR

SEQ ID NO: 139 - protein, 4 aa, synthetic construct
  REGION 1..4: note = Synthetic
RLTR

SEQ ID NO: 140 - protein, 4 aa, synthetic construct
  REGION 1..4: note = Synthetic
RKAR

SEQ ID NO: 141 - protein, 4 aa, synthetic construct
  REGION 1..4: note = Synthetic
RAQR

SEQ ID NO: 142 - protein, 4 aa, synthetic construct
  REGION 1..4: note = Synthetic
RQMR

SEQ ID NO: 143 - protein, 8 aa, synthetic construct
  REGION 1..8: note = Synthetic
RFVRRSNR

SEQ ID NO: 144 - protein, 4 aa, synthetic construct
  REGION 1..4: note = Synthetic
RQPR

SEQ ID NO: 145 - protein, 4 aa, synthetic construct
  REGION 1..4: note = Synthetic
CVRR

SEQ ID NO: 146 - protein, 4 aa, synthetic construct
  REGION 1..4: note = Synthetic
RSYR

SEQ ID NO: 147 - protein, 4 aa, synthetic construct
  REGION 1..4: note = Synthetic
IKKR

SEQ ID NO: 148 - protein, 4 aa, synthetic construct
  REGION 1..4: note = Synthetic
RHVR

SEQ ID NO: 149 - protein, 4 aa, synthetic construct
  REGION 1..4: note = Synthetic
RLAR

SEQ ID NO: 150 - protein, 4 aa, synthetic construct
  REGION 1..4: note = Synthetic
RPLR

SEQ ID NO: 151 - protein, 4 aa, synthetic construct
  REGION 1..4: note = Synthetic
IGKR

SEQ ID NO: 152 - protein, 4 aa, synthetic construct
  REGION 1..4: note = Synthetic
VTKR

SEQ ID NO: 153 - protein, 4 aa, synthetic construct
  REGION 1..4: note = Synthetic
RRHR

SEQ ID NO: 154 - protein, 8 aa, synthetic construct
  REGION 1..8: note = Synthetic
RRNRVHKR

SEQ ID NO: 155 - protein, 4 aa, synthetic construct
  REGION 1..4: note = Synthetic
RCPR

SEQ ID NO: 156 - protein, 4 aa, synthetic construct
  REGION 1..4: note = Synthetic
RWPR

SEQ ID NO: 157 - protein, 4 aa, synthetic construct
  REGION 1..4: note = Synthetic
SKER

SEQ ID NO: 158 - protein, 4 aa, synthetic construct
  REGION 1..4: note = Synthetic
RIKK

SEQ ID NO: 159 - protein, 4 aa, synthetic construct
  REGION 1..4: note = Synthetic
RIFR

SEQ ID NO: 160 - protein, 4 aa, synthetic construct
  REGION 1..4: note = Synthetic
RVWR

SEQ ID NO: 161 - protein, 4 aa, synthetic construct
  REGION 1..4: note = Synthetic
LEGR

SEQ ID NO: 162 - protein, 4 aa, synthetic construct
  REGION 1..4: note = Synthetic
LKKR

SEQ ID NO: 163 - protein, 4 aa, synthetic construct
  REGION 1..4: note = Synthetic
VFRR

SEQ ID NO: 164 - protein, 4 aa, synthetic construct
  REGION 1..4: note = Synthetic
IRKR

SEQ ID NO: 165 - protein, 4 aa, synthetic construct
  REGION 1..4: note = Synthetic
RQKR

SEQ ID NO: 166 - protein, 4 aa, synthetic construct
  REGION 1..4: note = Synthetic
RTKR

SEQ ID NO: 167 - protein, 4 aa, synthetic construct
  REGION 1..4: note = Synthetic
LQKR

SEQ ID NO: 168 - protein, 4 aa, synthetic construct
  REGION 1..4: note = Synthetic
KTRR

SEQ ID NO: 169 - protein, 4 aa, synthetic construct
  REGION 1..4: note = Synthetic
KTKR

SEQ ID NO: 170 - protein, 4 aa, synthetic construct
  REGION 1..4: note = Synthetic
KTRR